(19) United States
(12) Patent Application Publication
(10) Pub. No.: US 2025/0255671 A1
(43) Pub. Date: Aug. 14, 2025

(54) SYSTEM AND METHOD FOR PROVIDING ADJUSTABLE FORCE CONTROL FOR POWERED SURGICAL INSTRUMENTS

(71) Applicants: Smith & Nephew, Inc., Memphis, TN (US); Smith & Nephew Orthopaedics AG, Zug (CH); Smith & Nephew Asia Pacific PTE. Limited, Singapore (SG)

(72) Inventors: Ashley A. Roakes, Memphis, TN (US); Russell John Brooke, Memphis, TN (US); Mouhsin A. El-Chafei, Arlington, TN (US)

(73) Assignees: Smith & Nephew, Inc., Memphis, TN (US); Smith & Nephew Orthopaedics AG, Zug (CH); Smith & Nephew Asia Pacific PTE. Limited, Singapore (SG)

(21) Appl. No.: 18/724,380

(22) PCT Filed: Jan. 13, 2023
(86) PCT No.: PCT/US2023/010743

§ 371 Date: Jun. 26, 2024

Related U.S. Application Data
(60) Provisional application No. 63/303,718, filed on Jan. 27, 2022.

(51) Int. Cl. A61B 34/10; A61B 34/00; A61B 34/20; A61B 34/35

(52) U.S. Cl. CPC A61B 34/10; A61B 34/20; A61B 34/25; A61B 34/35; A61B 2034/105; A61B 2034/107; A61B 2034/2055; A61B 2034/258

(57) ABSTRACT

Disclosed herein is a system and method for generating a 3D map of a bone of a patient undergoing a total joint arthroplasty showing various volumes of the bone having different bone quality and/or patient characteristics and predicting, for each volume, a force threshold. The force threshold is used to automatically regulate the power output of a powered surgical tool depending on which volume of risk the tool is in contact with.

### RELATED APPLICATIONS

This application claims the benefit of U.S. Provisional Patent Application No. 63/303,718, filed Jan. 27, 2022, entitled “Adjustable Force Control for Powered Surgical Instruments”, the contents of which are incorporated herein in their entirety.

### FIELD OF THE DISCLOSURE

The present disclosure relates generally to orthopedic procedures, for example, total joint replacement procedures, and, in particular, to such procedures using powered surgical instruments.

### BACKGROUND

Orthopedic procedures to replace joints, for example, knees and hips, are well known and commonplace in today's society. Such procedures may require removal or reshaping of portions of a bone to accept an orthopedic implant to replace the original joint. During such procedures, a surgeon may use a powered surgical tool, for example, a powered impaction device or a powered bone burr, to remove or reshape the bone or to assist in the placement of the implant. Such procedures may be performed with the assistance of a navigated or robotic-assisted surgical platform which may track the position of the powered surgical tool with respect to the surgical theater or to the anatomy of the patient, and to provide guidance to the surgeon during the procedure.

For total joint arthroplasty (TJA) procedures, patients are at varying amounts of risk for adverse outcomes including, for example, intra-operative fractures and high rates of revision. Currently surgeons may incorporate information to modify the pre-operative surgical planning process to minimize the risk of fractures, given various data about the patient, for example, the patient's ancestry, lower bone density, thickness, etc. Currently, the implementation of the risk minimization focuses on restricting the spaces where powered surgical tools are active. However, it would be desirable to further modify the operation of the powered surgical tools to control and vary the power output of the tool, for example, the joules produced by an impaction tool or the RPM of a rotating tool, to further minimize the risk of an adverse outcome.

Systems currently exist that visualize the bone density of a patient as volumes of different colors and, in addition, teach a robotic system having a robotic arm configured with a power tool that does not allow the tool to leave a designated 3D volume. However, no systems are known which modifies performance of an automated or actively maintained surgical tool (referred to herein as a “powered tool” or a “powered surgical tool”) to minimize risk of intra-operative and post-operative adverse outcomes of the patient.

### SUMMARY OF THE DISCLOSURE

It is well known that outcomes of surgical procedures may be affected by many factors, including, for example, patient ancestry, anatomical features, biomechanical data, demographic information, and bone characteristics, such as bone density. For example, it has been reported that revision rates for total joint arthroplasty procedures differ across ancestry populations. Providing surgeons with this type of information about the patient can improve outcomes. For example, providing the surgeon information about the patient's bone characteristics allows them to work around as well as work within the limits for the quality of the bone, thereby reducing the risk of adverse outcomes, such as intra-operative fractures and high rates of revision.

The operation of powered surgical tools can be controlled based on various factors to both guide and control the position of the powered surgical tool but also to modify the output force and/or speed of powered cutting and impaction surgical tools, based on a map of the patient's characteristics as well as the patient's bone quality and predicted force thresholds.

The present disclosure incorporates an algorithm into a robotic solution that automatically modifies the power output of a powered surgical tool to remain below a patient's predicted force threshold (above which would increase the risk of adverse outcomes) to provide surgeons better control when performing a total joint arthroplasty or other orthopedic surgery.

In some examples, the present disclosure uses anatomical features of the patient, biomechanical data, bone characteristics and/or clinical data of the patient, for example, demographic data ancestry information, comorbidities, medical history, family history, genetics, genomics, and/or proteomics information, as input to an artificial intelligence model, for example, a machine learning model or computer algorithm which generates a 3D map of various volumes of the bone having different densities and, in some examples, predicts force thresholds for the various volumes of the bone based on the patient's bone quality.

In some examples, the 3D map is used to generate a non-navigated pre-operative plan that highlights steps of the surgical procedure where the surgeon has the highest risk of inducing a fracture based on the patient's data. As the surgeon performs the surgery, they are aware of which steps carry the highest risk of fracture and can prioritize extra focus during highlighted steps of the procedure.

In some examples, the 3D map is used to automatically adjust the output force or speed of a powered surgical tool in a navigated robotic platform during surgery to minimize the risk of intraoperative bone fracture complications.

In some examples, the 3D map is used to regulate powered impaction tools during surgery by changing the force and/or speed based on the predicted impact location on the bone relative to the location and orientation of patient's bone independent of a navigated robotic platform.

In a first example, a method comprises generating a 3D map of a bone, determining one or more risk volumes within the 3D map and predicting, for each risk volume, a force threshold.

In the first example, the method further comprises wherein the force threshold for each risk volume indicates a force output by a powered surgical tool that, if exceeded, has a high probability of causing a negative surgical outcome.

In the first example, or in any other example disclosed herein, the method further comprises wherein each risk volume is a volume indicating a consistent bone density.

In the first example, or in any other example disclosed herein, the method further comprises wherein the force threshold for each risk volume is predicted by an artificial intelligence model.

In the first example, or in any other example disclosed herein, the method further comprises wherein the artificial intelligence model is trained on a dataset which includes one or more of anatomical features the patient, biomechanical data of the patient, clinical data of the patient and bone characteristics of the patient.

In the first example, or in any other example disclosed herein, the method further comprises wherein the artificial intelligence model classifies each risk volume into a discrete force threshold category.

In the first example, or in any other example disclosed herein, the method further comprises generating a pre-operative plan identifying high-risk steps of a surgical procedure based on the 3D bone map and force thresholds.

In the first example, or in any other example disclosed herein, the method further comprises wherein the high-risk steps are determined algorithmically.

In the first example, or in any other example disclosed herein, the method further comprises advising a surgeon of the high-risk steps.

In the first example, or in any other example disclosed herein, the method further comprises modulating power output of a powered surgical tool using the force threshold associated with a risk volume with which the powered surgical tool is in contact.

In the first example, or in any other example disclosed herein, the method further comprises monitoring the position of the powered surgical tool during surgery to determine when the powered surgical tool moves from a first risk volume to a second risk volume.

In the first example, or in any other example disclosed herein, the method further comprises adjusting the power output of the powered surgical tool in accordance with a force threshold associated with the second risk volume.

In a second example, a system comprises a processor and software that, when executed by the processor causes the system to generate a 3D map of a bone, determine one or more risk volumes within the 3D map and predict, for each risk volume, a force threshold.

In the second example, or in any other example disclosed herein, the system further comprises wherein the force threshold for each risk volume is predicted by an artificial intelligence model.

In the second example, or in any other example disclosed herein, the system further comprises wherein the artificial intelligence model classifies each risk volume into a discrete force threshold category.

In the second example, or in any other example disclosed herein, the software further causes the system to advise a surgeon of the high-risk steps.

In the second example, or in any other example disclosed herein, the software further causes the system to modulate power output of a powered surgical tool using the force threshold associated with a risk volume with which the powered surgical tool is in contact.

In the second example, or in any other example disclosed herein, the software further causes the system to monitor a position of the powered surgical tool to determine when the powered surgical tool moves from a first risk volume to a second risk volume.

In the second example, or in any other example disclosed herein, the software further causes the system to adjust the power output of the powered surgical tool in accordance with a force threshold associated with the second risk volume.

In the second example, or in any other example disclosed herein, the system further comprises a display and wherein the software further causes the system to display the 3D map of the bone differentiating the risk volumes.

In the second example, or in any other example disclosed herein, the system further comprises wherein the powered surgical tool is an impaction tool and further wherein the force threshold is a maximum number of joules output by the powered surgical tool for a risk volume associated with the force threshold.

In the second example, or in any other example disclosed herein, the system further comprises wherein the powered surgical tool is a rotary tool and further wherein the force threshold is a maximum number of revolutions per minute output by the powered surgical tool for a risk volume associated with the force threshold.

In the second example, or in any other example disclosed herein, the system further comprises wherein the force threshold for each risk volume indicates a force output by a powered surgical tool that, if exceeded, has a high probability of causing a negative surgical outcome.

### BRIEF DESCRIPTION OF THE DRAWINGS

By way of example, specific examples of the disclosed system and method will now be described, with reference to the accompanying drawings, in which:

FIG. 1 depicts an operating theatre including an illustrative computer-assisted surgical system (CASS) in accordance with the described examples of the present disclosure.

FIG. 2 is a block diagram showing the artificial intelligence model of the described examples of the present disclosure.

FIG. 3 is an exemplary screenshot from the intra-operative software tool.

FIG. 4 is a block diagram showing the pre-operative example of the present disclosure.

FIG. 5 is a flowchart showing a method for regulating the power output of a powered surgical tool in accordance with the second example of the present disclosure.

### DEFINITIONS

For the purposes of this disclosure, the term “implant” is used to refer to a prosthetic device or structure manufactured to replace or enhance a biological structure. For example, in a total hip replacement procedure a prosthetic acetabular cup (implant) is used to replace or enhance a patients' worn or damaged acetabulum. While the term “implant” is generally considered to denote a man-made structure (as contrasted with a transplant), for the purposes of this specification an implant can include a biological tissue or material transplanted to replace or enhance a biological structure.

Although much of this disclosure refers to surgeons or other medical professionals by specific job title or role, nothing in this disclosure is intended to be limited to a specific job title or function. Surgeons or medical professionals can include any doctor, nurse, medical professional, or technician. Any of these terms or job titles can be used interchangeably with the user of the systems disclosed herein unless otherwise explicitly stated. For example, a reference to a surgeon could also apply, in some examples to a technician or nurse.

### DETAILED DESCRIPTION

The system and method disclosed herein provides information to a surgeon during the pre-operative and intra-operative phases of a joint replacement surgery which will assist the surgeon in planning and executing the surgery such as to minimize both intra-operative and post-operative negative outcomes for the patient. Note that, although the present disclosure is explained in terms of a total hip replacement procedure, all or portions of the present disclosure may be applicable to any joint replacement procedure.

This disclosure is not limited to the particular systems, devices and methods described, as these may vary. The terminology used in the description is for the purpose of describing the particular versions or examples only and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. As used in this document, the term “comprising” means “including, but not limited to”.

### CASS Overview

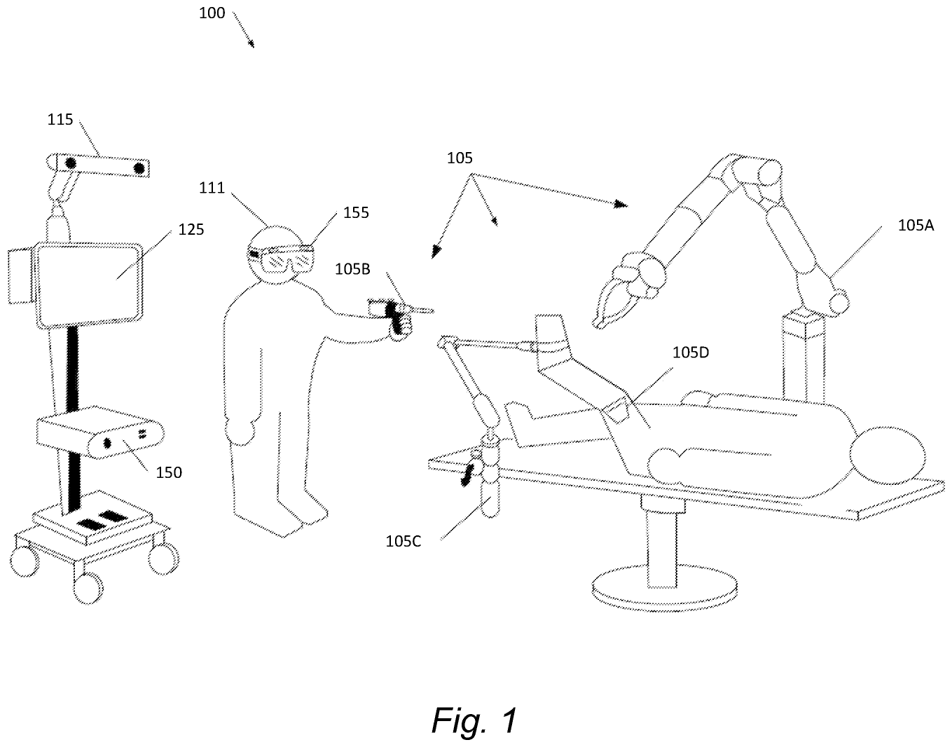

FIG. 1 provides an illustration of an example computer-assisted surgical system (CASS) 100, according to some examples. As described in further detail in the sections that follow, the CASS uses computers, robotics, and imaging technology to aid surgeons in performing orthopedic surgery procedures such as total knee arthroplasty (TKA) or total hip arthroplasty (THA). For example, surgical navigation systems can aid surgeons in locating patient anatomical structures, guiding surgical instruments, and implanting medical devices with a high degree of accuracy. Surgical navigation systems such as the CASS 100 often employ various forms of computing technology to perform a wide variety of standard and minimally invasive surgical procedures and techniques. Moreover, these systems allow surgeons to more accurately plan, track and navigate the placement of instruments and implants relative to the body of a patient, as well as conduct pre-operative and intra-operative body imaging.

An effector platform 105 positions surgical tools relative to a patient during surgery. The exact components of the effector platform 105 will vary, depending on the example employed. For example, for a knee surgery, the effector platform 105 may include an end effector 105B that holds surgical tools or instruments during their use. The end effector 105B may be a handheld device or instrument used by the surgeon (e.g., a NAVIO® hand piece or a cutting guide or jig) or, alternatively, the end effector 105B can include a device or instrument held or positioned by a robotic arm 105A.

The effector platform 105 can include a limb positioner 105C for positioning the patient's limbs during surgery. In some examples of a limb positioner 105C is the SMITH AND NEPHEW SPIDER2 system. The limb positioner 105C may be operated manually by the surgeon or alternatively change limb positions based on instructions received from the surgical computer 150 (described below).

The effector platform 105 can also include a cutting guide or jig 105D that is used to guide saws or drills used to resect tissue during surgery. Such cutting guides 105D can be formed integrally as part of the effector platform 105 or robotic arm 105A or cutting guides can be separate structures that can be matingly and/or removably attached to the effector platform 105 or robotic arm 105A. The effector platform 105 or robotic arm 105A can be controlled by the CASS 100 to position a cutting guide or jig 105D adjacent to the patient's anatomy in accordance with a pre-operatively or intraoperatively developed surgical plan such that the cutting guide or jig will produce a precise bone cut in accordance with the surgical plan.

The tracking system 115 uses one or more sensors to collect real-time position data that locates the patient's anatomy and surgical instruments. For example, for TKA procedures, the tracking system may provide a location and orientation of the end effector 105B during the procedure. In addition to positional data, data from the tracking system 115 can also be used to infer velocity/acceleration of anatomy/instrumentation, which can be used for tool control. In some examples, the tracking system 115 may use a tracker array attached to the end effector 105B to determine the location and orientation of the end effector 105B. The position of the end effector 105B may be inferred based on the position and orientation of the tracking system 115 and a known relationship in three-dimensional space between the tracking system 115 and the end effector 105B. Various types of tracking systems may be used in various examples of the present disclosure including, without limitation, Infrared (IR) tracking systems, electromagnetic (EM) tracking systems, video or image based tracking systems, and ultrasound registration and tracking systems.

Any suitable tracking system can be used for tracking surgical objects and patient anatomy in the surgical theatre. For example, a combination of IR and visible light cameras can be used in an array. Various illumination sources, such as an IR LED light source, can illuminate the scene allowing three-dimensional imaging to occur. In some examples, this can include stereoscopic, tri-scopic, quad-scopic, etc. imaging. In addition to the camera array, which in some examples is affixed to a cart, additional cameras can be placed throughout the surgical theatre. For example, handheld tools or headsets worn by operators/surgeons can include imaging capability that communicates images back to a central processor to correlate those images with images captured by the camera array. This can give a more robust image of the environment for modeling using multiple perspectives. Furthermore, some imaging devices may be of suitable resolution or have a suitable perspective on the scene to pick up information stored in quick response (QR) codes or barcodes. This can be helpful in identifying specific objects not manually registered with the system.

In some examples, specific objects can be manually registered by a surgeon with the system preoperatively or intraoperatively. For example, by interacting with a user interface, a surgeon may identify the starting location for a tool or a bone structure. By tracking fiducial marks associated with that tool or bone structure, or by using other conventional image tracking modalities, a processor may track that tool or bone as it moves through the environment in a three-dimensional model.

In some examples, certain markers, such as fiducial markers that identify individuals, important tools, or bones in the theater may include passive or active identifiers that can be picked up by a camera or camera array associated with the tracking system. For example, an IR LED can flash a pattern that conveys a unique identifier to the source of that pattern, providing a dynamic identification mark. Similarly, one or two dimensional optical codes (barcode, QR code, etc.) can be affixed to objects in the theater to provide passive identification that can occur based on image analysis. If these codes are placed asymmetrically on an object, they can also be used to determine an orientation of an object by comparing the location of the identifier with the extents of an object in an image. For example, a QR code may be placed in a corner of a tool tray, allowing the orientation and identity of that tray to be tracked. Other tracking modalities are explained throughout. For example, augmented reality headsets can be worn by surgeons and other staff to provide additional camera angles and tracking capabilities.

In addition to optical tracking, certain features of objects can be tracked by registering physical properties of the object and associating them with objects that can be tracked, such as fiducial marks fixed to a tool or bone. For example, a surgeon may perform a manual registration process whereby a tracked tool and a tracked bone can be manipulated relative to one another. By impinging the tip of the tool against the surface of the bone, a three-dimensional surface can be mapped for that bone that is associated with a position and orientation relative to the frame of reference of that fiducial mark. By optically tracking the position and orientation (pose) of the fiducial mark associated with that bone, a model of that surface can be tracked with an environment through extrapolation.

The registration process that registers the CASS 100 to the relevant anatomy of the patient can also involve the use of anatomical landmarks, such as landmarks on a bone or cartilage. For example, the CASS 100 can include a 3D model of the relevant bone or joint and the surgeon can intraoperatively collect data regarding the location of bony landmarks on the patient's actual bone using a probe that is connected to the CASS. Bony landmarks can include, for example, the medial malleolus and lateral malleolus, the ends of the proximal femur and distal tibia, and the center of the hip joint. The CASS 100 can compare and register the location data of bony landmarks collected by the surgeon with the probe with the location data of the same landmarks in the 3D model. Alternatively, the CASS 100 can construct a 3D model of the bone or joint without pre-operative image data by using location data of bony landmarks and the bone surface that are collected by the surgeon using a CASS probe or other means. The registration process can also include determining various axes of a joint. For example, for a TKA the surgeon can use the CASS 100 to determine the anatomical and mechanical axes of the femur and tibia. The surgeon and the CASS 100 can identify the center of the hip joint by moving the patient's leg in a spiral direction (i.e., circumduction) so the CASS can determine where the center of the hip joint is located.

The display 125 provides graphical user interfaces (GUIs) that display images and other information relevant to the surgery. For example, the display 125 overlays image information collected from various modalities (e.g., CT, MRI, X-ray, fluorescent, ultrasound, etc.) collected pre-operatively or intraoperatively to give the surgeon various views of the patient's anatomy as well as real-time conditions. The display 125 may include, for example, one or more computer monitors. As an alternative or supplement to the display 125, one or more members of the surgical staff may wear an augmented reality (AR) head mounted device (HMD). For example, in FIG. 1 the surgeon 111 is wearing an AR HMD 155 that may, for example, overlay pre-operative image data on the patient or provide surgical planning suggestions. Various example uses of the AR HMD 155 in surgical procedures are detailed in the sections that follow.

Surgical computer 150 provides control instructions to various components of the CASS 100, collects data from those components, and provides general processing for various data needed during surgery. In some examples, the surgical computer 150 is a general purpose computer. In other examples, the surgical computer 150 may be a parallel computing platform that uses central processing units (CPUs), graphics processing units (GPU), tensor processing units (TPU) or multiple computer instances in a cluster to perform processing. In some examples, the surgical computer 150 is connected to a remote server over one or more computer networks (e.g., the Internet). The remote server can be used, for example, for storage of data or execution of computationally intensive processing tasks.

Various techniques generally known in the art can be used for connecting the surgical computer 150 to the other components of the CASS 100. Moreover, the computers can connect to the surgical computer 150 using a mix of technologies. For example, the end effector 105B may connect to the surgical computer 150 over a wired (i.e., serial) connection. The tracking system 115, tissue navigation system 120, and display 125 can similarly be connected to the surgical computer 150 using wired connections. Alternatively, the tracking system 115, tissue navigation system 120, and display 125 may connect to the surgical computer 150 using wireless technologies such as, without limitation, Wi-Fi, Bluetooth, Near Field Communication (NFC), or ZigBee.

In some examples, CASS 100 may include a robotic arm 105A that serves as an interface to stabilize and hold a variety of instruments used during the surgical procedure. For example, in the context of a hip surgery, these instruments may include, without limitation, retractors, a sagittal or reciprocating saw, the reamer handle, the cup impactor, the broach handle, and the stem inserter. The robotic arm 105A may have multiple degrees of freedom (like a Spider device) and have the ability to be locked in place (e.g., by a press of a button, voice activation, a surgeon removing a hand from the robotic arm, or other method).

In some examples, movement of the robotic arm 105A may be effectuated by use of a control panel built into the robotic arm system. For example, a display screen may include one or more input sources, such as physical buttons or a user interface having one or more icons, that direct movement of the robotic arm 105A. The surgeon or other healthcare professional may engage with the one or more input sources to position the robotic arm 105A when performing a surgical procedure.

A tool or an end effector 105B, for example, a powered surgical tool, attached or integrated into a robotic arm 105A may include, without limitation, a burring device, a scalpel, a cutting device, a retractor, a joint tensioning device, or the like. In examples in which an end effector 105B is used, the end effector may be positioned at the end of the robotic arm 105A such that any motor control operations are performed within the robotic arm system. In examples in which a tool is used, the tool may be secured at a distal end of the robotic arm 105A, but motor control operation may reside within the tool itself.

The robotic arm 105A may be motorized internally to both stabilize the robotic arm, thereby preventing it from falling and hitting the patient, surgical table, surgical staff, etc., and to allow the surgeon to move the robotic arm without having to fully support its weight. While the surgeon is moving the robotic arm 105A, the robotic arm may provide some resistance to prevent the robotic arm from moving too fast or having too many degrees of freedom active at once. The position and the lock status of the robotic arm 105A may be tracked, for example, by a controller or the Surgical Computer 150.

In some examples, the robotic arm 105A can be moved by hand (e.g., by the surgeon) or with internal motors into its ideal position and orientation for the task being performed. In some examples, the robotic arm 105A may be enabled to operate in a “free” mode that allows the surgeon to position the arm into a desired position without being restricted. While in the free mode, the position and orientation of the robotic arm 105A may still be tracked as described above. In some examples, certain degrees of freedom can be selectively released upon input from user (e.g., surgeon) during specified portions of the surgical plan tracked by the Surgical Computer 150. Designs in which a robotic arm 105A is internally powered through hydraulics or motors or provides resistance to external manual motion through similar means can be described as powered robotic arms, while arms that are manually manipulated without power feedback, but which may be manually or automatically locked in place, may be described as passive robotic arms.

A robotic arm 105A or end effector 105B can include a trigger or other means to control the power of a saw or drill. Engagement of the trigger or other means by the surgeon can cause the robotic arm 105A or end effector 105B to transition from a motorized alignment mode to a mode where the saw or drill is engaged and powered on. Additionally, the CASS 100 can include a foot pedal (not shown) that causes the system to perform certain functions when activated. For example, the surgeon can activate the foot pedal to instruct the CASS 100 to place the robotic arm 105A or end effector 105B in an automatic mode that brings the robotic arm or end effector into the proper position with respect to the patient's anatomy in order to perform the necessary resections. The CASS 100 can also place the robotic arm 105A or end effector 105B in a collaborative mode that allows the surgeon to manually manipulate and position the robotic arm or end effector into a particular location. The collaborative mode can be configured to allow the surgeon to move the robotic arm 105A or end effector 105B medially or laterally, while restricting movement in other directions. As discussed, the robotic arm 105A or end effector 105B can include a cutting device (saw, drill, and burr) or a cutting guide or jig 105D that will guide a cutting device. In other examples, movement of the robotic arm 105A or robotically controlled end effector 105B can be controlled entirely by the CASS 100 without any, or with only minimal, assistance or input from a surgeon or other medical professional. In still other examples, the movement of the robotic arm 105A or robotically controlled end effector 105B can be controlled remotely by a surgeon or other medical professional using a control mechanism separate from the robotic arm or robotically controlled end effector device, for example using a joystick or interactive monitor or display control device.

### Description of Examples

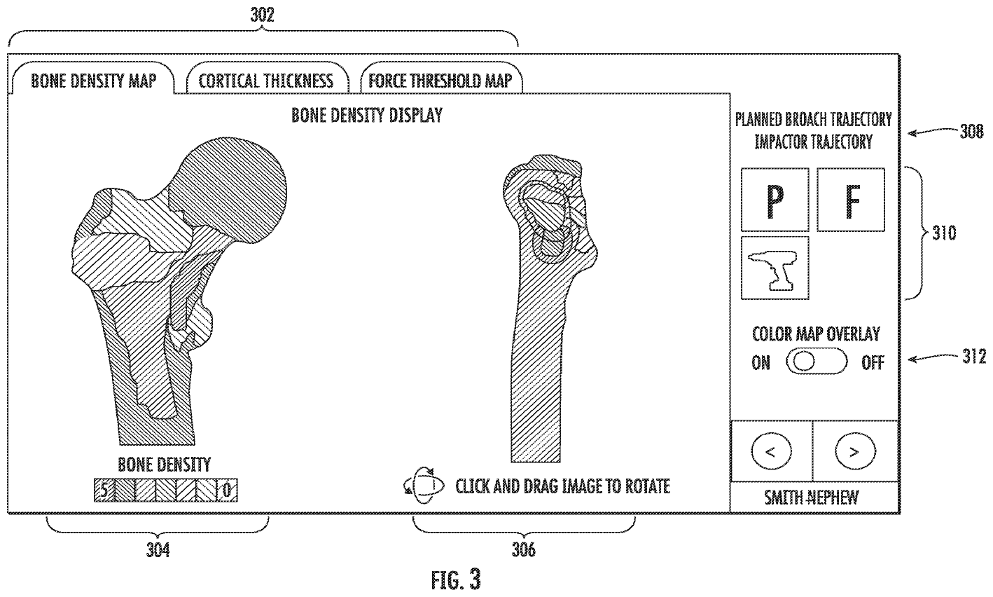

All examples of the present disclosure described herein are dependent upon the generation of a three-dimensional (3D) map of a bone of the patient, wherein the three-dimensional map is provided with an overlay showing volumes of the bone having varying risks associated with the surgical procedure, referred to herein as “risk volumes”. For example, each risk volume could indicate a volume of the bone having a different bone density. In this case, bone densities will vary from risk volume to risk volume. In other examples, each risk volume could indicate a characteristic of the volume of bone other than bone density. The map may be generated utilizing data from a bone scan or other medical imaging technologies including X-ray, CT and/or MRI performed on the patient. In some examples, the risk volumes are depicted in different colors, although any method of differentiating the risk volumes from each other may be used. An example of such a map is shown as reference numbers 304, 306 in FIG. 3, showing the femur of a patient during a total hip arthroplasty procedure, wherein the risk volumes are shown in different colors.

In some examples of the present disclosure, each risk volume is assigned a force threshold. The force thresholds are indicative of the maximum power that should be output by a powered surgical tool 105B during the surgical procedure when the powered surgical tool 105B is in contact with the associated risk volume. For example, for an impaction tool, the force threshold may be indicative of a maximum number of joules that should be output by the impaction tool for a particular risk volume. Likewise, for a rotary tool, the force threshold could be expressed as a maximum number of revolutions per minute of the tool. The force threshold indicates a force output by the power tool which, if exceeded, has a high probability of causing a negative outcome for the patient, for example, an intra-operative fracture of the bone or a stressing of the bone such as to cause a likelihood of the need for revision surgery in the future.

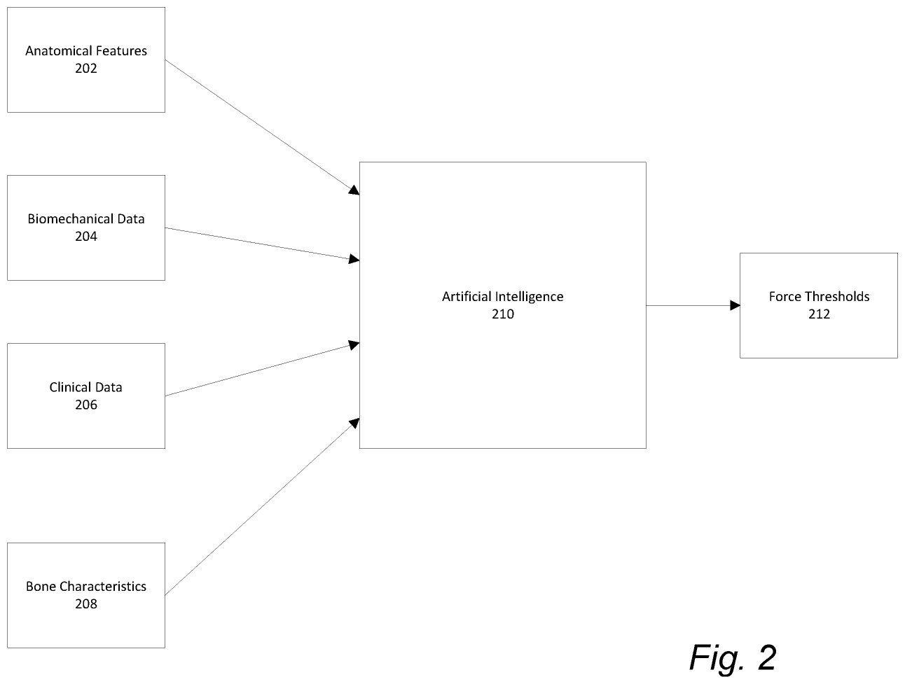

In some examples, the force thresholds for each risk volume may be generated by an artificial intelligence model. A block diagram of an exemplary artificial intelligence model is shown in FIG. 2. The artificial intelligence model 210 is preferably implemented as a machine learning model comprising one or more convolutional neural networks having forward and backward propagation to optimize the weights of the model. In other examples, other forms of machine learning models or artificial intelligence may be used. In preferred examples, the artificial intelligence model may be trained on a training dataset comprising various types of information about the patient.

The training dataset may include bone characteristic data 208. The bone characteristic data 208 may include, for example, one or more of the medical imaging technologies previously mentioned, bone measurement information and bone classification information. For example, in the instance where the surgical procedure is a total hip arthroplasty, the bone classification information can include a Dorr classification of the patient's femur.

The training dataset may further include clinical data 206. The clinical data 206 may comprise demographic information about the patient (including, for example, age, sex, weight, height, etc.), ancestry information, medical history, family history, comorbidities, genetics, genomics and/or proteomics information. Additionally, the clinical data 206 may also include a metric indicating the activity level of the patient and information regarding the region of the world in which the patient was born or resides. Any other demographic-type information may also be used.

The training data set may further include biomechanical data 204. The biomechanical data 204 may include, for example, one or both of information about the patient's muscles and information from synthetic models of the patient's anatomy.

Lastly, the training data set may further include anatomical features 202 of the patient, including, for example, one or both of measurements of the patient's anatomy and a 3D bone model of the bones of the patient involved in the surgical procedure.

The training data set for artificial intelligence model 210, in various examples, may include any combination of anatomical features 202, biomechanical data 204, clinical data 206, and/or bone characteristics 208, or any other information germane to the procedure. In the training phase, the metrics regarding the force threshold for each density volume may be generated using finite element analysis or other methods of generating the thresholds mathematically.

As the artificial intelligence model 210 is used in the testing phase, the actual forces used in the surgical procedure and the post-operative insights of the surgeon regarding a positive or negative outcome of the surgery may be fed back into artificial intelligence model 210 to improve the accuracy of the model. Also, in the testing phase, any combination of available data may be input to artificial intelligence model 210. For example, patients of a particular sex and age may commonly have areas of a particular bone which are typically less dense in patients of that sex and age. In the absence of a medical imaging scan to the artificial intelligence model 210, the clinical data 206 could be used on a generic model of the bone.

The output of artificial intelligence model 210, in preferred examples of the present disclosure, comprises a force threshold 212 for each risk volume of the bone of interest. The force thresholds may comprise a generic metric which may be applied to any model of the powered surgical tool or may be metric specific to a particular powered surgical tool.

In a first example of the present disclosure, the 3D map of the bone having the overlay indicating the risk volumes 304, 306, as well as the force thresholds for each risk volume may be used in a pre-operative context. In the pre-operative context, the information may be used to highlight the steps of the surgical procedure exhibiting an elevated risk of inducing a fracture. This information may allow the surgeon to plan the surgical procedure and to prioritize extra focus during the identified steps of the procedure.

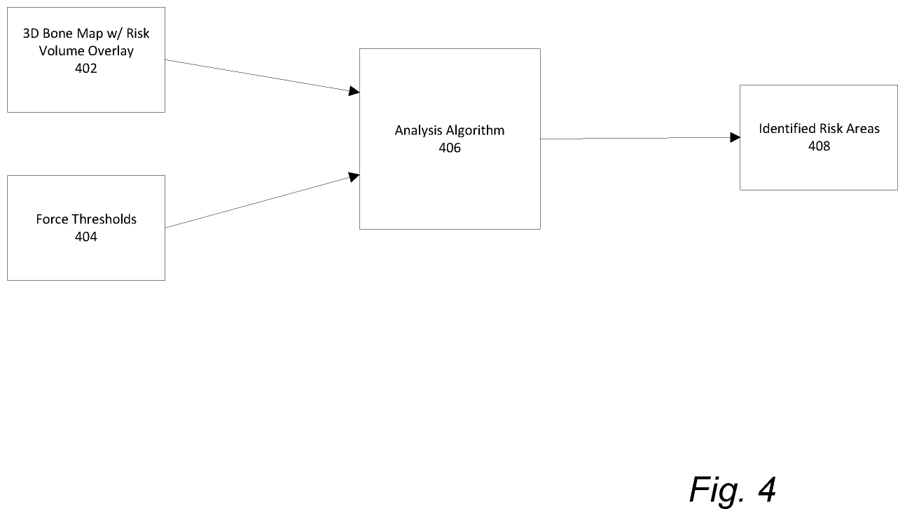

FIG. 4 shows a block diagram of the pre-operative use of the present disclosure. The analysis algorithm 406 may take as input the 3D bone map with the risk volume overlay 402 as well as the force thresholds 404 generated by artificial intelligence model 210 and outputs the identified risk areas 408.

In a second example of the present disclosure, the 3D map of the bone 304, 306 having the overlay indicating the risk volumes, as well as the force thresholds 212 for each risk volume may be used intraoperatively to minimize the risk of bone fracture complications and to improve the confidence of the surgeon in the procedure.

In one variation of the second example, the 3D map of the bone 304, 306 may be displayed on display 125. An exemplary example of a screen displaying the 3D map 304, 306 is shown in FIG. 3. In this particular exemplary example, the risk volumes are indicative of bone density, as indicated by the “Bone Density Display” indicia and the “Bone Density Map” tab displayed on the exemplary screen. The exemplary screen may include tabs 302 to display other information besides the bone density map 304, 306, for example, a display showing the cortical thickness (not shown) or a display showing the force threshold map (not shown). In addition, the exemplary screen may provide, during a total hip arthroplasty procedure, and indicator 308 of the planned broach trajectory and the impactor trajectory which may be also overlaid on the 3D map of the bone 304, 306. In the exemplary screen shown in FIG. 3, indicators 310 indicate that marker arrays connected to the patient's pelvis, the patient's femur, and the powered surgical tool are visible to tracking system 115. In the event that tracking system 115 loses contact with any of the marker arrays, the respective button may be displayed in a different color, for example, red. Toggle switch 312 allows the surgeon to remove the color display of the risk volumes and such that only the 3D map of the bone is displayed.

The exemplary screen shown in FIG. 3 may be displayed on display 125 and may updated in real-time during the surgical procedure to provide the surgeon with dynamic feedback as the procedure progresses. This allows the surgeon to know when the powered surgical tool is in contact with a particular risk volume such that the surgeon may be aware of the higher risk involved with particular risk volumes.

In another variation of the second example, the information regarding the force thresholds may be incorporated into an algorithm in CASS 100 that automatically modifies the power output of the powered surgical tool 105B such that the power output remains below the patient's predicted force threshold for a particular risk volume. Exceeding the force threshold may increase the risk of an adverse outcome. In certain examples, the 3D map 304, 306 and the force thresholds may also be used to regulate a powered impaction tool 105B during surgery by changing the speed or force based on the predicted impact location on the bone relative to the location and orientation of patient's bone.

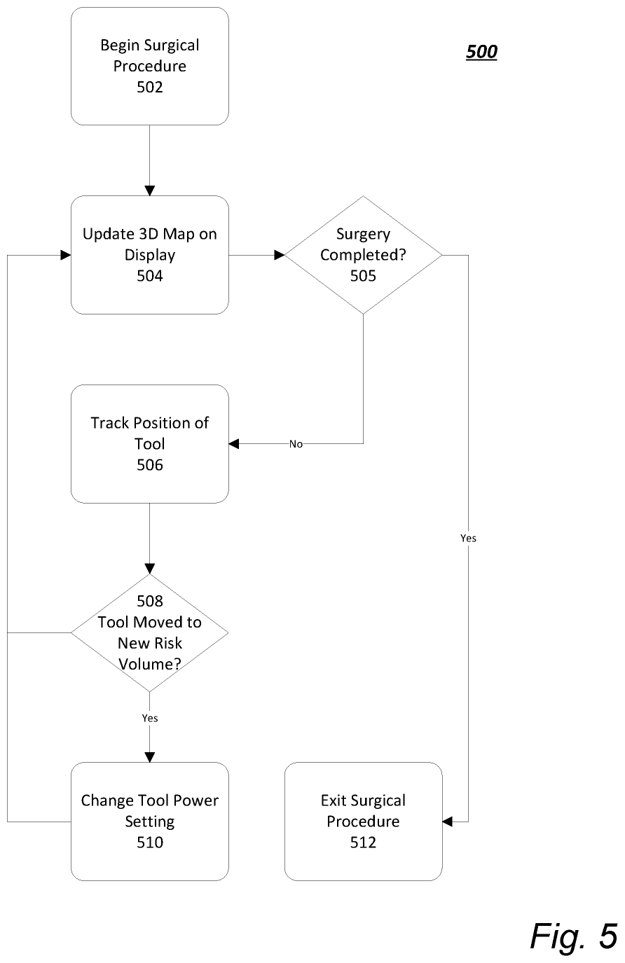

FIG. 5 is a flowchart showing the operation of the algorithm for modifying the power output of powered surgical tool 105B intraoperatively. The surgical procedure begins at 502 and, at 504 the 3D map displayed on display 125 is updated. At 505, if the surgery is complete, the surgical procedure ends at 512 and if the surgery is not complete, control proceeds to box 506, where the position of the powered surgical tool 105B is tracked. At 508 is determined if the powered surgical tool 105B has moved to a new risk volume and, if so, the power output of powered surgical tool 105B is changed in step 510 in accordance with the force threshold associated with the new risk volume. After it is determined if the power output of the powered surgical tool 105B needs to be modified, control returns to 504, where the map is updated to reflect the current position of the powered surgical tool 105B. The loop proceeds in the same manner until the end of the surgery is reached at 512.

In preferred examples of the present disclosure, the software executing on surgical computer 150 may be modified to incorporate the algorithm which controls the power of powered surgical tool 105B. In addition, the software may be further altered to display, on display 125, the exemplary screen of FIG. 3 showing the 3D map of the bone 304, 306 having the risk volumes overlaid thereon. In the first example involving the pre-operative planning stage, the software implementing the algorithm for determining the high risk areas of the surgical procedure may be implemented on surgical computer 150 or maybe implemented on an independent system, for example, a desktop computer, a laptop computer or a computing tablet.

In the above detailed description, reference is made to the accompanying drawings, which form a part hereof. In the drawings, similar symbols typically identify similar components, unless context dictates otherwise. The illustrative examples described in the present disclosure are not meant to be limiting. Other examples may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular examples described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular examples only and is not intended to be limiting.

We claim:

1. A method comprising:
generating a 3D map of a bone;
determining one or more risk volumes within the 3D map; and
predicting, for each risk volume, a force threshold.

2. The method of claim 1, wherein the force threshold for each risk volume indicates a force output by a powered surgical tool that, if exceeded, has a high probability of causing a negative surgical outcome.

3. The method of claim 1, wherein each risk volume of the bone is a volume indicating a consistent bone density.

4. The method of claim 1, wherein the force threshold for each risk volume is predicted by an artificial intelligence model.

5. The method of claim 4, wherein the artificial intelligence model is trained on a dataset which includes one or more of anatomical features the patient, biomechanical data of the patient, clinical data of the patient and bone characteristics of the patient.

6. The method of claim 4, wherein the artificial intelligence model classifies each risk volume into a discrete force threshold category.

7. The method of claim 1, further comprising:
generating a pre-operative plan identifying high-risk steps of a surgical procedure based on the 3D bone map and force thresholds.

8. The method of claim 7, wherein the high-risk steps are determined algorithmically.

9. The method of claim 7, further comprising advising a surgeon of the high-risk steps.

10. The method of claim 1, further comprising:
modulating power output of a powered surgical tool using the force threshold associated with a risk volume with which the powered surgical tool is in contact.

11. The method of claim 10, further comprising:
monitoring a position of the powered surgical tool to determine when the powered surgical tool moves from a first risk volume to a second risk volume.

12. The method of claim 11, further comprising:
adjusting the power output of the powered surgical tool in accordance with a force threshold associated with the second risk volume.

13. A system comprising:
a processor; and
software that, when executed by the processor, causes the system to:
generate a 3D map of a bone;
determine one or more risk volumes within the 3D map; and
predict, for each risk volume, a force threshold.

14. The system of claim 13, wherein the force threshold for each risk volume is predicted by an artificial intelligence model.

15. The system of claim 14, wherein the artificial intelligence model classifies each risk volume into a discrete force threshold category.

16. The system of claim 13, wherein the software further causes the system to:
generate a pre-operative plan identifying high-risk steps of a surgical procedure, based at least on the identified risk volumes.

17. The system of claim 16, wherein the software further causes the system to:
advise a surgeon of the high-risk steps.

18. The system of claim 13, wherein the software further causes the system to:
modulate power output of a powered surgical tool using the force threshold associated with a risk volume with which the powered surgical tool is in contact.

19. The system of claim 18, wherein the software further causes the system to:
monitor a position of the powered surgical tool to determine when the powered surgical tool moves from a first risk volume to a second risk volume.

20. The system of claim 19, wherein the software further causes the system to:
adjust the power output of the powered surgical tool in accordance with a force threshold associated with the second risk volume.

21. The system of claim 13, further comprising:
a display;
wherein the software further causes the system to:
display the 3D map of the bone differentiating the risk volumes.

22. The system of claim 18, wherein the powered surgical tool is an impaction tool and further wherein the force threshold is a maximum number of joules output by the powered surgical tool for a risk volume associated with the force threshold.

23. The system of claim 18, wherein the powered surgical tool is a rotary tool and further wherein the force threshold is a maximum number of revolutions per minute output by the powered surgical tool for a risk volume associated with the force threshold.

24. The system of claim 13, wherein the force threshold for each risk volume indicates a force output by a powered surgical tool that, if exceeded, has a high probability of causing a negative surgical outcome.
